(19) United States
(12) Patent Application Publication
(10) Pub. No.: US 2004/0254112 A1
(43) Pub. Date: Dec. 16, 2004

(54) Use of pulmonary surfactant for the early treatment of acute pulmonary diseases

(76) Inventor: Dietrich Hafner, Konstanz (DE)

Correspondence Address: Todd L. Juneau, NATH & ASSOCIATES PLLC, 6th Floor, 1030 15th Street, N.W., Washington, DC, 20005, US

(21) Appl. No.: 10/837,604

(22) Filed: May 4, 2004

Related U.S. Application Data
(63) Continuation-in-part of application No. 10/240,324, filed on Mar. 6, 2003.
Application No. 10/240,324 is a 371 of international application No. PCT/EP01/04223, filed on Apr. 12, 2001.

(30) Foreign Application Priority Data
Apr. 12, 2000 (EP) 00107858.3

(51) Int. Cl. A61K 38/17

(52) U.S. Cl. 514/12

(57) ABSTRACT

The invention describes the use of pulmonary surfactant preparations for the early treatment of acute pulmonary diseases.

### TECHNICAL FIELD OF THE INVENTION

The invention relates to the use of pulmonary surfactant preparations for the early treatment of acute pulmonary diseases.

### PRIOR ART

ARDS (Adult Respiratory Distress Syndrome) is a descriptive expression which is applied to a large number of acute, diffuse infiltrative pulmonary lesions of differing etiology if they are associated with a severe gas exchange disorder (in particular arterial hypoxemia) (G. R. Bernard et al.: Report of the American-European consensus conference on ARDS: definitions, mechanisms, relevant outcomes and clinical trial coordination; Intensive Care Medicine, 1994, 20:225-232). The expression ARDS is used because of numerous common clinical and pathological features as seen with IRDS (Infant Respiratory Distress Syndrome). In case of IRDS, a lung surfactant deficiency caused by premature birth is predominant. In case of ARDS, the lung surfactant malfunction Is caused by the disease of the lung which is based on differing etiologies. Triggering causes for an ALI (Acute Lung Injury) including ARDS can, for example, be (cited in accordance with Harrison's Principles of Internal Medicine 10th Ed. 1983 McGraw-Hill Int Book Comp.) diffuse pulmonary infections (e.g. due to viruses, bacteria, fungi), aspiration of, for example, gastric juice or in the case of near-drowning, inhalation of toxins or irritants (e.g. chlorine gas, nitrogen oxides, smoke), direct or indirect trauma (e.g. multiple fractures or pulmonary contusion), systemic reactions to inflammations outside the lung (e.g. hemorrhagic pancreatitis, gram-negative septicemia), transfusions of high blood volumes or alternatively after cardiopulmonary bypass.

Surfactant abnormalities of differing severity are also reported for a number of other disease conditions, for example in obstructive pulmonary disorders such as asthma, bronchiolitis, COPD (Chronic Obstructive Pulmonary Disease) and after lung transplantation or alternatively after cardiopulmonary bypass (survey, see, for example, M. Griese Eur. Respir. J. 1999; 13: 1455-1467). Macnaughton et al. (Chest 1994; 106: 421425) and DoCampo et al. (Lancet 1994; 343: 482) describe the administration of exogenous surfactant after cardiopulmonary bypass. McBrien et al. (Lancet 1993, 342:1485-1486) and Suzuki et al. (Eur. J. Pediatr. 1996; 155: 383-384) describe the administration of surfactant after near-drowning. Struber et al. (Cardiovasc. Surg. 1995; 110; 563-564) describe the administration of surfactant after lung transplantation.

Presently, therapy of ARDS consists mainly in the earliest possible application of different forms of ventilation [e.g. PEEP (positive end-expiratory pressure), raising of the oxygen concentration of the respiratory air, SIMV (Synchronized Intermittent Mandatory Ventilation; Harrison's Principles of Internal Medicine 10th Ed. 1983 McGraw-Hill Int Book Comp)] up to extracorporeal membrane oxygenation (ECMO; Zapol and Lemaire Adult Respiratory Distress Syndrome, Marcel Dekker Inc. 1991). The specific use of various ventilation techniques has only led to a small lowering of mortality and includes the risk of setting in motion a vicious circle. By ventilation with pressure and high FiO₂ (Fraction of Inspired Oxygen; proportion of oxygen in the respiratory air), the lungs themselves can be damaged and as a result of this even higher pressures and higher FiO₂ may be required in order to obtain an adequate oxygenation of the blood. For many years, it has proven suitable to treat IRDS by introducing pulmonary surfactant preparations into the lungs of the children concerned.

It is known in the art, that surfactant preparations can be used in the treatment of IRDS or ARDS.

WO9204907 refers to the use of a lipid composition of bovine pulmonary surfactant plus proteins SP-B and/or SP-C to prevent and treat neonatal respiratory distress syndrome (IRDS) or adult respiratory distress syndrome (ARDS). WO9849191 refers to surfactant compositions such as surfactant compositions including KL4 peptide, BLES, Infrasurf, Alveofact, Surfactant TA, Survanta, Curosurf, Exosurf or Alec for the treatment of respiratory distress syndrome, removal of inflammatory exudates from inflamed lung tissues, or the treatment of meconium aspiration syndrome in infants.

Häfner et al. (Häfner et al. (1999) Pulmonary Pharmacology & Therapeutics 12: 193) describe the effects of early treatment with rSP-C surfactant on oxygenation and histology in rats with acute lung injury, i.e. its effects on preventing hyaline membrane formation in the rat lungs.

WO0027360 refers to a set comprising a first container, which contains a diluted pulmonary surfactant preparation, and a second container, which contains a pulmonary surfactant preparation. According to WO0027360 pulmonary surfactant preparations may contain phospholipids and pulmonary surfactant proteins such as SP-B, SP-C or modified derivatives thereof, in particular rSP-C. WO0027360 also refers to a method for the treatment or prophylaxis of ALI including ARDS, the treatment or prophylaxis of IRDS, the treatment or prophylaxis of pneumonia, bronchitis, meconium aspiration syndrome, COPD, asthma and cystic fibrosis with dilute pulmonary surfactant solution and subsequent administration of pulmonary surfactant.

It is known from pilot studies that pulmonary surfactant preparations are clinically active in ALI including ARDS (survey, for example, B. Lachmann, D. Gommers and E. P. Eijking: Exogenous surfactant therapy in adults, Atemw.-Lungenkrkh. 1993, 19:581-91; D. Walmrath et al.: Bronchoscopic surfactant administration in patients with severe adult respiratory distress syndrome and sepsis, Arm. J. Respir. Crit Care Med. 1996, 154:5762: T. J. Gregory et al.: Bovine surfactant therapy for patients with acute respiratory distress syndrome, Am. J. Respir. Crit. Care Med. 1997. 155:1309-16).

Walmrath D et al. (Walmrath D et al. (2002) Eur Respir J 19: 805-810) describe the use of a biovine surfactant preparation in the treatment of patients suffering from severe ARDS. According to Walmrath et al., patients had been ventilated for no longer than 96 hours since ARDS diagnosis and after a stable baseline period of additional 6 hours the bovine surfactant preparation was bronchoscopically administered.

Spragg R G et al. (Spragg R G et al. (2003) Am J Respr Care Med 167: 1562) also describe a late treatment of ARDS patients. According to Spragg et al., patients who had ARDS as defined by American European Consensus Conference (20) criteria for not more than 48 hours, i.e. 36.5±8.2 hours (High Group (1 ml of rSP-C)) and 28.8±3.6 (Low Group (0.5 ml rSP-C)), were subjected to pulmonary surfactant preparation treatment The study results revealed that no significant treatment benefit was associated with this late surfactant treatment.

Thus, it is known in the art that in clinical trials surfactant preparations are applied to ARDS patients late in the treatment, i.e. later than 24 hours after diagnosing the ARDS.

Finally, it is also known in the art (Textbook of Respiratory Medicine. Ed. Murray, Nadel: Mason, Boushey. W B Saunders Company 2000, pages 2414 to 2415) that in the early phase of acute lung injury, which is within 7 days following the specific lung injury (see. FIG. 92.2, page 2415), pulmonary edema and its effects are most pronounced and that therefore manipulations to affect the rate of edema genesis are most likely to have an impact.

### DESCRIPTION OF THE INVENTION

The object of the present invention is the provision of a treatment method of acute pulmonary diseases. Surprisingly, it has now been found that pulmonary surfactant preparations, in particular those which contain recombinant surfactant protein C, are suitable for the early treatment of acute pulmonary diseases in humans. As a result of the early treatment of acute pulmonary diseases by administration of a pulmonary surfactant preparation in humans In need thereof the therapeutic benefit of such an early treatment is significantly improved compared to a late treatment of acute pulmonary disease as known in the art Thus, for example, the treatment benefit associated with the early treatment of ALI or ARDS patients with a pulmonary surfactant preparation measurable as the frequency of adverse events or as the gas exchange rate or as the number of ventilator-free days is significantly improved compared to patients treated with a pulmonary surfactant preparation late after diagnosis of ALI or ARDS. As a consequence, the stay of patients in intensive care units (i.e. the number of days) can be shortened and thus costs can be saved. Furthermore, in patients who are ventilated, it is possible by the administration of pulmonary surfactant preparation to avoid side effects of ventilation, for example the risk of a nosocomial infection or pneumonia.

In a first aspect, the invention therefore relates to a method of early treating acute pulmonary diseases in a human in need thereof comprising the step of administering to said human a therapeutically effective amount of a pulmonary surfactant preparation.

According to the invention, the term “early treatment” of acute pulmonary diseases in humans in need thereof is understood as meaning the treatment of humans who are in the stage of development of an acute lung disease. In particular, the term early treatment of ARDS patients refers to the treatment of ARDS patients as defined by the American and European Consensus Conference (Bernard G R et al. (1994) Am J Respir Crit care Med 149: 818-824) who have ARDS for not more than 24 hours. It is preferred that early treatment of ARDS patents refers to the treatment of ARDS patients as defined by the American and European Consensus Conference (Bernard G R et al. (1994) Am J Respir Crit care Med 149: 818-824) who have ARDS for not more than 16 hours. It is particularly preferred that early treatment of ARDS patients refers to the treatment of ARDS patients as defined by the American and European Consensus Conference (Bernard G R et al. (1994) Am J Respir Crit care Med 149: 818-824) who have ARDS for not more than 8 hours

A further subject of the invention is a method of early treating ARDS patients who have ARDS for not more than 24 hours, in particular for not more than 16 hours, more particular for not more than 8 hours, by administering to said ARDS patients a therapeutically effective amount of a pulmonary surfactant preparation, in particular a pulmonary surfactant preparation containing recombinant surfactant protein C.

According to the invention, the term “prophylaxis” of acute lung diseases in mammals is understood as meaning the complete or partial prevention (i.e. attenuation) of an acute lung disease, in particular in mammals which are not yet suffering from the acute lung disease or whose predisposing factors allow such a development to be assumed.

Exemplary “acute lung diseases” according to the invention are ALI, ARDS, acute respiratory insufficiency, pneumonias (in particular ventilation-induced pneumonias), nosocomial infections or SIRS (systemic inflammatory response syndrome) associated with ALI.

According to the invention, “humans in need” are preferably human patients with a diagnosed acute lung disease. In particular, these are patients with diagnosed ALI or ARDS, patients with diagnosed acute respiratory insufficiency, patients with diagnosed pneumonia, patients with diagnosed nosocomial infection, patients with hypothermia or patients with SIRS associated with ALI. By way of example, patients selected from the following patient groups may be mentioned: patients before, during or after intervention on the open thorax, patients who are ventilated, patients with pulmonary intoxication, patients with trauma, patents with sepsis, patients with pneumonia or those in which the risk of pneumonia exists, patients with a nosocomial infection or in which the risk of a nosocomial Infection exists, patients with hypothermia and patients with SIRS associated with ALI.

In a further aspect of present invention, there is provided a method of early treating acute pulmonary diseases in human patents with diagnosed acute pulmonary disease selected from the group consisting of ALI, ARDS, acute respiratory insufficiency, pneumonia, nosocomial infection, hypothermia and SIRS associated with ALI, the method comprises the step of administering to said human patients a therapeutically effective amount of a pulmonary surfactant preparation.

Natural pulmonary surfactant has surface-active properties; it reduces, for example, the surface tension in the alveoli. A simple and rapid in vitro test with which the surface activity of pulmonary surfactant can be determined is, for example, the so-called Wilhelmy balance [Goerke, J. Biochim. Biophys. Acta, 344: 241-261 (1974), King R. J. and Clements J. A. Am. J. Physicol. 223: 715-726 (1972)]. This method gives information on the pulmonary surfactant quality, measured as the action of a pulmonary surfactant of achieving a surface tension of almost zero mN/nm. Another measuring device for determining the surface activity of pulmonary surfactant is the pulsating bubble surfactometer [Possmayer F., Yu S. and Weber M., Prog. Resp. Res., Ed. v. Wichert, Vol. 18: 112-120 (1984)].

The activity of a pulmonary surfactant preparation can also be determined by means of in vivo tests, for example as described by Häfner et al. (D. Häfner et al.: Effects of rSP-C surfactant on oxygenation and histology in a rat lung lavage model of acute lung injury. Am. J. Respir. Crit Care Med. 1998, 158: 270-278). By the measurement of, for example, the pulmonary compliance, the blood gas exchange or the ventilation pressures needed, It is possible to obtain information on the activity of a pulmonary surfactant.

“Pulmonary surfactant preparation” is understood according to the invention as meaning the numerous known compositions and their modifications that have the function of natural pulmonary surfactant. In this case, preferred compositions are those which, for example, have activity in the tests described above. Particularly preferred compositions are those, which exhibit increased activity in such a test in comparison with natural, in particular human, pulmonary surfactant. In this context, these can be compositions that only contain phospholipids, but also compositions, which, apart from the phospholipids, inter alia additionally contain pulmonary surfactant protein. Preferred phospholipids according to the invention are dipalmitoylphosphatidylcholine (DPPC), palmitoyloleylphosphatidyl-glycerol (POPG) and/or phosphatidylglycerol (PG). Particularly preferably, the phospholipids are mixtures of various phospholipids, in particular mixtures of dipalmitoylphosphatidylcholine (DPPC) and palmitoyloleylphosphatidylglycerol (POPG), preferably in the ratio from 7 to 3 to 3 to 7. Commercial products that may be mentioned are Curosurf® (Serono, Pharma GmbH, Unterschleiβheim), a natural surfactant from homogenized porcine lungs, Survanta® (Abbott GmbH, Wiesbaden) and Alveofact® (Boehringer Ingelheim), both extracts of bovine lungs, as well as Exosurf® (Glaxo Wellcome), a synthetic phospholipid containing excipients. Suitable pulmonary surfactant proteins are both the proteins obtained from natural sources, such as pulmonary lavage or extraction from amniotic fluid, and the proteins prepared by genetic engineering or chemical synthesis. According to the invention, in particular the pulmonary surfactant proteins designated by SP-B and SP-C and their modified derivatives are of interest. The amino acid sequences of these pulmonary surfactant proteins, their isolation or preparation by genetic engineering are known (e.g. from WO 86/03408, EP-A 0251449, WO-89/04326, WO 87/06943, WO 88/03170, WO 91/00871, EP-A 0368823 and EP-A-0 348 967). Modified derivatives of the pulmonary surfactant proteins designated by SP-C, which differ from human SP-C by the replacement of a few amino acids, are described, for example, in WO 91/18015 and WO 95/32992. Particularly to be emphasized in this connection are the recombinant SP-C derivatives which are disclosed in WO 95/32992, in particular those which differ from human SP-C in positions 4 and 5 by the replacement of cysteine by phenylalanine and in position 32 by the replacement of methionine by isoleucine [designated below as rSP-C (FF/I) or lusupultide (INN)]. Modified derivatives of pulmonary surfactant proteins are also understood as meaning those proteins which have a completely originally designed amino acid sequence with respect to their pulmonary surfactant properties, such as are described in EP-A 0593094 and WO 92122315. Preferably, the polypeptde KL4 (INN: sinapultide) may be mentioned in this connection. The name pulmonary surfactant protein, according to the invention, also comprises mixtures of different pulmonary surfactant proteins. In EP-B 0100910, EP-A 0110498, EP-B 0119056, EP-B 0145005 and EP-B 0286011, phospholipid compositions with and without pulmonary surfactant proteins are described which are likewise suitable as components of the preparations.

In a further aspect of present invention there Is provided a method of early treating acute pulmonary diseases in a human in need thereof comprising the step of administering to said human a therapeutically effective amount of a pulmonary surfactant preparation comprising phospholipids and lusupultide, if desired together with further excipients.

As further constituents, which can be present in pulmonary surfactant preparations, fatty acids such as palmitic acid may be mentioned. The pulmonary surfactant preparations can also contain electrolytes such as calcium, magnesium and/or sodium salts (for example calcium chloride, sodium chloride and/or sodium hydrogencarbonate) in order to establish an advantageous viscosity. Preferred preparations according to the invention contain 80 to 95% by weight of phospholipids, 0.5 to 3.0% by weight of pulmonary surfactant proteins, 3 to 15% by weight of fatty acid, preferably palmitic acid, and 0 to 3% by weight of calcium chloride.

Therefore, a further aspect of present invention is a method of early treating acute pulmonary diseases in a human in need thereof comprising the step of administering to said human a therapeutically effective amount of a pulmonary surfactant preparation which contains 80 to 95% by weight of phospholipids, 0.5 to 3.0% by weight of pulmonary surfactant proteins, 3 to 15% by weight of fatty acid, preferably palmitic acid, and 0 to 3% by weight of calcium chloride.

The pulmonary surfactant preparations are prepared by processes known per se and familiar to the person skilled in the art, for example as described in WO 95/32992. According to the invention, the pulmonary surfactant preparations are preferably lyophilized and in particular spray-dried pulmonary surfactant preparations. Lyophilized preparations are disclosed, for example, in WO 97/35882, WO 91/00871 and DE 3229179. WO 97/26863 describes a process for the preparation of powdered pulmonary surfactant preparations by spray drying. According to the invention, preparations prepared in this way are preferred.

The patients having interventions on the open thorax can be, according to the invention, patients in which an intervention is carried out on the heart, such as a bypass operation or a heart valve operation. Furthermore, they can be patients in which an intervention is carried out on the lung, such as a lung transplantation or a pneumonectomy. In connection with the treatment of patents with lung transplantations, according to the invention a preliminary treatment of the organ to be transplanted with pulmonary surfactant preparation is preferably carried out before the transplantation, in particular before the storage of the transplant, particularly preferably before removal of the transplant from the organ donor. Novick et al. (Evaluation of Surfactant Treatment Strategies after Prolonged Graft Storage in Lung Transplantation; Am. J. Respir. Crit. Care Med. 1996, Vol. 154, 98-104) describe surfactant treatment strategies in connection with storage of lung transplants. Preferably, the patient who receives the organ donation is pretreated before transplantation with pulmonary surfactant preparation. After transplantation has taken place, further treatment of the patient with the pulmonary surfactant preparation can then be carried out

According to the invention, ventilation of a patient is understood as meaning ventilation of the lungs which is brought about by aid or artificial respiration. An exemplary aid for ventilation which may be mentioned is the respirator, where different forms of ventilation known to the person skilled in the art can be used. Patients who are ventilated are, in particular, patients where spontaneous respiration is absent or insufficient. By way of example, patients having respiratory insufficiency, patients having central or peripheral respiratory paralysis, patients having ventilation under anesthesia, patients having long-term ventilation in intensive medicine and patients who are ventilated in the course of resuscitation. The ventilation of patients contains the risk of damaging the lung (Ventilation-Induced Lung Injury; VILI) and setting a vicious circle in motion. As a result of ventilation using pressure and a high FiO₂ (Fraction of Inspired Oxygen; proportion of oxygen in the respiratory air), the lung itself can be damaged and this can have the result that even higher pressures and higher FiO₂ are needed in order to obtain adequate oxygenation of the blood. At the same time, in the case of mechanically ventilated patients an increased risk of pneumonias and nosocomial infections exists (Michael J. Richards et al. Critical Care Medicine 1999; 27:887-892). The prophylactic treatment of mechanically ventilated patients, in particular of those in which no ALI or ARDS is yet present, with pulmonary surfactant preparations can therefore lead to earlier weaning from the mechanical ventilation, lower the risk of setting a vicious circle in motion and additionally also decrease the risk of a nosocomial infection or pneumonia in such patients. The invention further also relates to a procedure for the mechanical ventilation of a patent, pulmonary surfactant preparation being administered to the mechanically ventilated patient. According to the invention, the patient Is preferably a patient who has not yet developed ALI or ARDS.

Patients having pulmonary intoxication can be, for example, patients having pulmonary intoxication as a result of a bone marrow transplantation (toxic lung injury after bone marrow transplantation), or patients having a pulmonary intoxication which was caused by toxic gases.

The patients having trauma are, according to the invention, in particular patients having thoracic, cranial or cerebral trauma or those having multiple traumas.

Patients having hypothermia are in particular patients having a lowered body temperature in the case of collapse, hypothyroidism, cachexia, accidental hypothermia due to exposure to cold (e.g. in mountain and drowning accidents) and controlled hypothermia as is used, for example, in open heart surgery, in neurosurgery and in transplantations.

In a further aspect of present invention there is provided a process for the early treatment of acute pulmonary diseases in humans in need, in particular of those patients with diagnosed ARDS or ALI. The process is characterized in that a therapeutically efficacious and pharmacologically tolerable amount of a pulmonary surfactant preparation, in particular a pulmonary surfactant preparation comprising recombinant surfactant protein C, is administered early in the course of the disease to the human in need, in particular early after diagnosing the diseases. The pulmonary surfactant preparation is administered not later than 24 hours after diagnosing ARDS (as defined by the American and European Consensus Conference; Bernard G R et al. (1994) Am J Respir Crit care Med 149: 818-824). Particularly, the pulmonary surfactant preparation is administered not later than 16 hours after diagnosing ARDS. It is particularly preferred that the pulmonary surfactant preparation is administered not later than 8 hours after diagnosing ARDS.

In a further aspect of present invention, there is provided a process for the early treatment of ARDS or ALI, the process is characterized in that in a first step ARDS or ALI Is diagnosed and in a second step within 24 hours after ARDS or ALI has been diagnosed a therapeutically efficacious and pharmacologically tolerable amount of a pulmonary surfactant preparation, in particular a pulmonary surfactant preparation comprising recombinant surfactant protein C, is administered to the human in need. The dosage of the pulmonary surfactant preparation is carried out in the order of magnitude customary for pulmonary surfactant preparation. According to the invention, the pulmonary surfactant preparation in the case of the early treatment of ALI or ARDS is preferably administered to the patient not later than 24 hours after diagnosing ALI or ARDS condition. The pulmonary surfactant preparation is administered in a manner known to the person skilled in the art, preferably by intratracheal Instillation (infusion or bolus) of a pulmonary surfactant solution or suspension or in the form of an atomization of a pulmonary surfactant solution or suspension or by atomization of pulmonary surfactant powder. Preferably, the preparations according to the invention for administration are dissolved or suspended in a suitable solvent or resuspension medium, in particular if the preparations are present in lyophilized or spray-dried form. Preferably, the suitable resuspension medium is a physiological saline solution. It has proven advantageous to administer suspensions or solutions of the preparations according to the invention which contain 12.5 to 100 mg of phospholipids per ml of suspension. Preferably, the preparations according to the invention are administered per application in such an amount that the amount of phospholipids is between 12.5 and 200 mg per kilogram of body weight. As a rule, administration is carried out 1 to 3 times daily over a period of 1 to 7 days. A process is preferred in which the pulmonary surfactant solution employed contains 0.5 to 2.0 mg of rSP-C (FF/1) per ml of solvent. Particular mention may be made of a process in which the pulmonary surfactant solution employed contains 0.75 to 1.5 mg of rSP-C (FF/1) per ml of solvent. If desired, before the administration of the preparations according to the invention a bronchoalveolar lavage, preferably with dilute pulmonary surfactant preparation, can be carried out. Such a procedure is described, for example, in Gommers et al. [Bronchoalveolar lavage with a diluted surfactant suspension prior to surfactant instillation improves the effectiveness of surfactant therapy in experimental acute respiratory distress syndrome (ARDS), Intensive Care Med. 1998, 24:494-500] and in WO 98/49191.

If desired, the pulmonary surfactant preparations can also be administered in combination with other medicaments in the early treatment of acute lung diseases, in particular with those medicaments which are customarily employed for the treatment of acute lung diseases.

### EXAMPLES

A.) Production of Powdered Pulmonary Preparations

Powdered pulmonary surfactant preparations are produced by the process described in WO 97/26863:

### Example 1

7.0 g of 1,2-dipalmitoyl-3-sn-phosphatidylcholine, 2.5 g of 1-palmitoyl-2-oleoyl-3-sn-phosphatidylglycerol sodium, 205 mg of calcium chloride dihydrate and 250 mg of palmitic acid are dissolved in 300 ml of ethanol/water (85:15) with warming to 60° C., cooled to room temperature and mixed with 350 ml of a solution of rSP-C (FF/I) in chloroform/methanol 9:1 (c=429 mgA). The resulting solution is spray-dried in a Büchi B 191 laboratory spray dryer. Spray conditions: drying gas air, inlet temperature 90° C., outlet temperature 52-54° C. A relatively loose powder is obtained.

### Example 2

A solution of the surfactant obtained from bovine lungs (obtained by extraction and purification steps such as described, for example, in EP 406732) in chloroform/methanol is spray-dried under the following conditions: Büchi B 191 laboratory spray dryer, drying gas nitrogen, inlet temperature 80° C., outlet temperature 50-52° C. A fine, yellowish powder is obtained.

### Example 3

10.95 g of 1,2-dipalmitoyl-3-sn-phosphatidylcholine, 4.6 g of 1-palmitoyl-2-oleoyl-3-sn-phosphatidylglycerol ammonium, 418 mg of calcium chloride dihydrate and 750 mg of palmitic acid are dissolved in 330 ml of 2-propanol/water (85:15) at 50° C. and, after cooling to 30° C., mixed with 620 ml of a solution of rSP-C (FF/I) in isopropanol/water (95:5, c=484 mg/l). The resulting solution is spray-dried in a Büchi B 191 laboratory spray dryer. Spray conditions: drying gas nitrogen, inlet temperature 100° C., outlet temperature 58-60° C. A colorless powder Is obtained.

### Example 4

3.74 g (5.1 mmol) of 1,2-dipalmitoyl-3-sn-phosphatidylcholine, 2.81 g (3.7 mmol) of 1-palmitoyl-2-oleoyl-3-sn-phosphatidylcholine, 2.90 g (3.9 mmol) of 1,2-dipalmitoylphosphatidyl-3-sn-phosphatidylglycerol sodium, 234 mg of palmitic acid and 279 mg (1.9 mmol) of calcium chloride dihydrate are dissolved in 160 ml of 2-propanol/water (85:15) at 50° C. and, after cooling to 30° C., mixed with 566 ml of a solution of rSP-C (FF/l) in isopropanol/water (92: 8, c=330 mg/l) at 30° C. The resulting solution is spray-dried in a Büchi B 191 laboratory spray dryer. Spray conditions: drying gas nitrogen, inlet temperature 90° C., outlet temperature 58-60° C. A colorless powder is obtained.

### Example 5

0.5 g of KL4 (INN; sinapultide), 7.125 g of 1,2-dipalmitoyl-3-sn-phosphatidylcholine and 2.43 g of 1-palmitoyl-2-oleoyl-3-sn-phosphatidylglycerol ammonium are dissolved in 500 ml of chloroform/methanol 1:1 with warming to 45° C. and then spray-dried In a Büchi B 191 laboratory spray dryer. Spray conditions: drying gas nitrogen, inlet temperature 85° C., outlet temperature 55° C. A colorless powder is obtained.

### Example 6

A solution of phospholipids, palmitic acid and calcium chloride dehydrate obtainable according to Example 1, 3 or 4 is spray-dried—without addition of a solution of rSP-C (FF/1)—corresponding to the conditions according to Example 1, 3 or 4. A powder is obtained.

B.) Production of the Medicaments According to the Invention

### Example 7

0.1 to 10 g of the powder obtained according to Example 1 are dispensed into a bottle of volume 100 to 250 ml and the bottle is sealed. The bottle is packed in a suitable folding box together with a pack insert.

### Example 8

3 g of the powder obtained according to Example 1 are poured into a bottle having a volume of 66 ml and the bottle is closed. The bottle is packed in a suitable folded box together with a pack insert.

C.) Surfactant Administration

### Example 9

Patients having ARDS as defined by the American and European Consensus Conference (Bernard G R et al. (1994) Am J Respir Crit care Med 149: 818-824) for not more than 24 hours received a rSP-C (lusupultide)-based surfactant preparation that contains (wt/wt) 1,8% rSP-C, 63,4% 2-dipalmitoyl-sn-s-phosphatidylcholine, 27,8% 1-palmitoyl-2-oleoyl-3-sn-phosphatidylglycerol, 4,5% palmitic acid, and 2,5% calcium chloride dihydrate. Patients received 1 ml of rSP-C surfactant (containing 1 mg of rSP-C and 50 mg of phospholipisds) per kilogram lean body weight up to four times in 24 hours. Surfactant was administered through a catheter inserted through the endotracheal tube.

### Example 10

Patients having ARDS as defined by the American and European Consensus Conference (Bernard G R et al. (1994) Am J Respir Crit care Med 149: 818-824) for not more than 24 hours received a rSP-C (lusupultide)-based surfactant preparation that contains (wt/wt) 1,8% rSP-C, 63,4% 2-dipalmitoyl-sn-s-phosphatidylcholine, 27,8% 1-palmitoyl-2-oleoyl-3-sn-phosphatidylglycerol, 4,5% palmitic acid, and 2,5% calcium chloride dihydrate. Patients received 0,5 ml of rSP-C surfactant (containing 0,5 mg of rSP-C and 24 mg of phospholipisds) per kilogram lean body weight up to four times in 24 hours. Surfactant was administered through a catheter inserted through the endotracheal tube.

1. A method of early treating acute pulmonary diseases in a human in need thereof comprising the step of administering to said human a therapeutically effective amount of a pulmonary surfactant preparation.

2. The method as claimed in claim 1, the pulmonary surfactant preparation comprising phospholipids, the pulmonary surfactant proteins SP-B and/or SP—C and/or their modified derivatives, if desired together with further excipients.

3. The method as claimed in claim 2, the pulmonary surfactant protein being recombinantly prepared pulmonary surfactant proteins.

4. The method as claimed in claim 2, the pulmonary surfactant protein being lusupultide.

5. The method as claimed in claims 2 to 4, the pulmonary surfactant preparation contains 80 to 95% by weight of phospholipids, 0,5 to 3,0% by weight of pulmonary surfactant proteins, 3 to 15% by weight of fatty acids, and 0 to 3% by weight of calcium chloride.

6. The method as claimed in claim 1, the human in need being a patient with a diagnosed acute pulmonary disease selected from the group consisting of ALI, ARDS, acute respiratory insufficiency, pneumonia, nosocomial infection, hypothermia and SIRS associated with ALI.

7. The method as claimed in claim 6, the patient being selected from the following patient groups: patients before, during or after an intervention on the open thorax, patients who are ventilated, patients with pulmonary intoxication, patients with a trauma, patients with sepsis, patients with pneumonia or those in which the risk of pneumonia exists, patients with a nosocomial Infection or in which the risk of a nosocomial infection exists, patients with hypothermia and patients with SIRS (systemic inflammatory response syndrome) associated with ALI.

8. The method as claimed in claim 6, the human in need being an ARDS patient having ARDS for not more than 24 hours.

9. The method as claimed in claim 8, the ARDS patient having ARDS for not more than 16 hours.

10. The method as claimed in claim 8, the ARDS patient having ARDS for not more than 8 hours.
